(19) United States
(12) Patent Application Publication
(10) Pub. No.: US 2007/0185172 A1
(43) Pub. Date: Aug. 9, 2007

(54) Organic compounds

(76) Inventors: David Andrew Sandham, West Sussex (GB); Christopher William Sharp, West Sussex (GB); Simon James Watson, West Sussex (GB)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/571,894

(22) PCT Filed: Jul. 13, 2005
(86) PCT No.: PCT/EP05/07634

§ 371 Date: Feb. 22, 2007

(30) Foreign Application Priority Data
Jul. 14, 2004 (GB) 0415747.5

(51) Int. Cl. A61K 31/41

(52) U.S. Cl. 514/359; 548/240

(57) ABSTRACT

Compounds of formula I 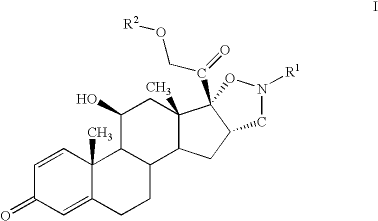
in free or salt form, wherein R¹ and R² have the meanings as indicated in the specification, are useful for treating inflammatory conditions, particularly inflammatory or obstructive airways diseases. Pharmaceutical compositions that contain the compounds and processes for preparing the compounds are also described.

This invention relates to organic compounds, their preparation and use as pharmaceuticals.

In a first aspect, the present invention provides compounds of formula I 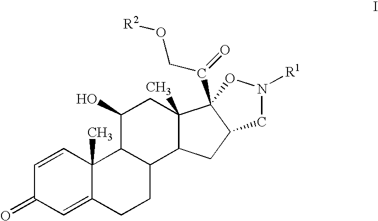
in free or salt form, wherein
R¹ is selected from the group consisting of C₁-C₈-alkyl and C₃-C₈-cycloalkyl; and
R² is selected from the group consisting of hydrogen, C₂-C₈-alkylcarbonyl and C₃-C₈-cycloalkylcarbonyl.

Terms used in the specification have the following meanings:

“C₁-C₈-alkyl” denotes straight chain or branched alkyl having 1 to 8 carbon atoms. Preferably C₁-C₈-alkyl is C₁-C₄-alkyl.

“C₃-C₈-cycloalkyl” denotes a cycloaliphatic group having 3 to 8 carbon atoms, for example cyclopropyl, methylcyclopropyl, cyclobutyl, methylcyclobutyl, cyclopentyl, cyclohexyl, methylcyclohexyl, dimethylcyclohexyl or cycloheptyl, but is preferably C₃-C₆-cycloalkyl.

“C₂-C₈-alkylcarbonyl” denotes carbonyl substituted by C₂-C₈-alkyl as hereinbefore defined. Preferably C₂-C₈-alkylcarbonyl is C₂-C₄-alkylcarbonyl.

“C₃-C₈-cycloalkylcarbonyl” denotes carbonyl substituted by a cycloaliphatic group having 3 to 8 carbon atoms, for example cyclopropyl-, methylcyclopropyl-, cyclobutyl-, methylcyclobutyl-, cyclopentyl-, cyclohexyl-, methylcyclohexyl-, dimethylcyclohexyl- or cycloheptyl- or cyclooctylcarbonyl, but is preferably C₃-C₆-cycloalkylcarbonyl.

Throughout this specification and in the claims that follow, unless the context requires otherwise, the word “comprise”, or variations such as “comprises” or “comprising”, will be understood to imply the inclusion of a stated integer or step or group of integers or steps but not the exclusion of any other integer or step or group of integers or steps.

Compounds of formula I are capable of forming acid addition salts, particularly pharmaceutically acceptable acid addition salts.

Pharmaceutically acceptable acid addition salts of the compound of formula I include those of inorganic acids, for example, hydrohalic acids such as hydrofluoric acid, hydrochloric acid, hydrobromic acid or hydroiodic acid, nitric acid, sulfuric acid, phosphoric acid; and organic acids, for example aliphatic monocarboxylic acids such as formic acid, acetic acid, trifluoroacetic acid, propionic acid and butyric acid, aliphatic hydroxy acids such as lactic acid, citric acid, tartaric acid or malic acid, dicarboxylic acids such as maleic acid or succinic acid, aromatic carboxylic acids such as benzoic acid, p-chlorobenzoic acid, diphenylacetic acid or triphenylacetic acid, aromatic hydroxy acids such as o-hydroxybenzoic acid, p-hydroxybenzoic acid, 1-hydroxynaphthalene-2-carboxylic acid or 3-hydroxynaphthalene-2-carboxylic acid, and sulfonic acids such as methanesulfonic acid or benzenesulfonic acid. These salts may be prepared from compounds of formula I by known salt-forming procedures.

Preferred compounds of formula I include those wherein,
R¹ is selected from the group consisting of C₁-C₈-alkyl and C3-C₈-cycloalkyl; and
R² is selected from the group consisting of hydrogen and C₂-C₈-alkylcarbonyl.

Especially preferred compounds of formula I include those wherein, either
R¹ is selected from the group consisting of C₁-C₄-alkyl and C₃-C₆-cycloalkyl, and
R² is selected from the group consisting of hydrogen and C₂-C₄-alkylcarbonyl.

More especially preferred compounds of formula I include those hereinafter described in the Examples.

In a second aspect, the present invention provides a process for the preparation of compounds of formula I as hereinbefore defined which comprises:
(i) (A) for the preparation of compounds of formula I wherein R² is hydrogen, hydrolysing a corresponding ester; or
(B) for the preparation of compounds of formula I wherein R² is selected from the group consisting of C₂-C₈-alkylcarbonyl and C₃-C₈-cycloalkylcarbonyl, appropriately acylating a corresponding compound of formula I wherein R² is hydrogen; and
(ii) recovering the resultant compounds of formula I in free or salt form.

Process variant (A) may be carried out using known procedures for hydrolysing esters to the corresponding alcohols. Conveniently, the ester is reacted with an aqueous solution of an inorganic carbonate, preferably potassium carbonate. The reaction is preferably carried out in a protogenic solvent, for example methanol. The reaction temperature is conveniently from 0° C. to ambient temperature.

Process variant (B) may be carried out using known procedures for acylating alcohols to provide the corresponding ester. Conveniently, the alcohol is reacted with an anhydride, for example isobutyric anhydride, in a basic solvent, for example pyridine, or a solvent that contains a base. The reaction temperature is conveniently from 0° C. to 35° C.

In a third aspect, the present invention provides novel intermediate compounds of formula II 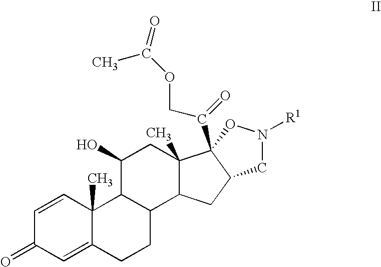
in free or salt form, wherein
R¹ is selected from the group consisting of C₂-C₈-alkyl and C₃-C₈-cycloalkyl.

These may be used as the starting ester in process variant (A).

Compounds of formula II may be prepared by reacting a compound of formula III 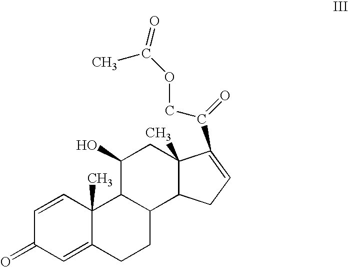
with a compound of formula IV 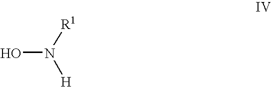
wherein R¹ is selected from the group consisting of C₂-C₈-alkyl and C₃-C₈-cycloalkyl, in the presence of paraformaldehyde and a base, preferably a secondary amine such as di-isopropyl amine, using known procedures for isoxazolidine formation by cycloaddition of a nitrone. The reaction is conveniently carried out in a protogenic solvent, for example ethanol. The reaction is conveniently carried out at an elevated temperature, for example from 60 to 85° C.

The compound of formula III is known and can be prepared from prednisolone according to the method detailed in Yoon et al, Steroids (1995) 60:445-451.

Compounds of formula IV are either commercially available or may be prepared according to the method detailed in Tetrahedron Letters (1987) 28: 2993-2994.

Compounds of formula I in free form may be converted into salt form, and vice versa, in a conventional manner. The compounds in free or salt form can be obtained in the form of hydrates or solvates containing a solvent used for crystallisation.

Compounds of formula I can be recovered from reaction mixtures and purified in a conventional manner.

Compounds of formula I are useful as pharmaceuticals. Accordingly, the invention also provides a compound of formula I for use as a pharmaceutical. The compounds of formula I have important pharmacological properties. For example, they have a high anti-inflammatory activity, which can be demonstrated by their binding to the glucocorticoid receptor and inhibition of TNF-alpha synthesis and release in a human macrophage cell line.

Binding to the glucocorticoid receptor may be measured in the following assay:

Recombinant human GR expressed in baculovirus-infected insect Sf-9 cells is obtained from Panvera (Madison, Wis., USA), as is GR assay buffer and the proprietary fluorescent ligand Fluormone™-GS1 (200 nM methanol solution). The assay is conducted in ₃₈₄ well plates by sequential addition of a serially diluted dimethylsulfoxide solution of test compound in water (2 μl), Fluormone™-GS1 (2.2 nM in GR assay buffer, 10 μl) and GR solution (8.8 nM in GR assay buffer, 10 μl). The assay is incubated in the dark at room temperature for 1 hr, prior to fluorescence polarisation measurement using an analyst multiwell instrument with 485 nm excitation and 530 nm emission filters. The concentration of test compound resulting in half-maximum shift in polarisation gives the IC₅₀. Curves are fitted using Origin™ software and Ki values were calculated using the Cheng-Prussoff equation. Compounds of Examples 1, 2, 3, and 4 have Ki values of 0.5, 1.4, 0.4 and 0.2 nM respectively in this assay.

Inhibition of TNF-alpha synthesis and release may be measured by the following assay:

Human macrophage cell line U937 is obtained from American Type Culture Collection (Rockville Md.) and cultured in RPMI 1640 (Gibco UK) supplemented with 10% FCS (Gibco UK). Cell density is adjusted to 4×10⁵ cells/ml and the cells are differentiated by adding phorbal myristate acetate (PMA, 20 ng/ml) for 4 hours. The PMA is removed by washing and the adherent cells are incubated for a further 48 hours at 37° C. in a humidified incubator with 5% CO₂. Differentiated U937 cells are removed using cell dissociation buffer (Gibco UK) and the cell density is adjusted to 1×10⁶ cells/ml. 100 μl of the cell suspension is placed in 96 well culture plates and 50 μl of either medium or compound at the appropriate concentration in dimethylsulfoxide are added. After a preincubation of 20 minutes at 37° C., the cells are stimulated with 10 ng/ml lipopolysaccharide (LPS, Sigma) and the supernatants are harvested after 24 hours of incubation at 37° C. in a humidified incubator with 5% CO₂. Concentration of TNFα in the supernatants is determined by sandwich ELISA using two monoclonal antibodies recognising different epitopes of the cytokine (Pharmingen UK). Binding of the second antibody is analysed by stepwise incubation with streptavidin alkaline phosphatase conjugate (Sigma UK) and 4-nitrophenylphosphate disodium salt. Optical density is measured at 405 nm and cytokine concentration calculated based on results from serial diluations of standard recombinant TNFα. Curves are fitted and IC₅₀ values calculated using Origin™ software.

Compounds of Examples 1, 2, 3, and 4 have IC₅₀ values of 1.16, 4.95, 0.89 and _{3.57} nM respectively in this assay.

Having regard to their anti-inflammatory activity, compounds of formula I are useful in the treatment of inflammatory conditions, particularly inflammatory or obstructive airways diseases. Treatment in accordance with the invention may be symptomatic or prophylactic.

Inflammatory or obstructive airways diseases to which the present invention is applicable include asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma induced following bacterial infection. Treatment of asthma is also to be understood as embracing treatment of subjects, e.g. of less than 4 or 5 years of age, exhibiting wheezing symptoms and diagnosed or diagnosable as “wheezy infants”, an established patient category of major medical concern and now often identified as incipient or early-phase asthmatics (For convenience this particular asthmatic condition is referred to as “wheezy-infant syndrome”).

Prophylactic efficacy in the treatment of asthma will be evidenced by reduced frequency or severity of symptomatic attack, e.g. of acute asthmatic or bronchoconstrictor attack, improvement in lung function or improved airways hyperreactivity. It may further be evidenced by reduced requirement for other, symptomatic therapy, i.e. therapy for or intended to restrict or abort symptomatic attack when it occurs, e.g. anti-inflammatory (e.g. corticosteroid) or bronchodilatory. Prophylactic benefit in asthma may in particular be apparent in subjects prone to “morning dipping”. “Morning dipping” is a recognised asthmatic syndrome, common to a substantial percentage of asthmatics and characterised by asthma attack, e.g. between the hours of about 4 to 6 a.m., i.e. at a time normally substantially distant form any previously administered symptomatic asthma therapy.

Other inflammatory or obstructive airways diseases and conditions to which the present invention is applicable include acute lung injury (ALI), adult/acute respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular other inhaled drug therapy. The invention is also applicable to the treatment of bronchitis of whatever type or genesis including, e.g., acute, arachidic, catarrhal, croupus, chronic or phthinoid bronchitis. Further inflammatory or obstructive airways diseases to which the present invention is applicable include pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis.

Having regard to their anti-inflammatory activity, compounds of formula I may also be useful in the treatment of eosinophil related disorders, e.g. eosinophilia, in particular eosinophil related disorders of the airways (e.g. involving morbid eosinophilic infiltration of pulmonary tissues) including hypereosinophilia as it effects the airways and/or lungs as well as, for example, eosinophil-related disorders of the airways consequential or concomitant to Löffler's syndrome, eosinophilic pneumonia, parasitic (in particular metazoan) infestation (including tropical eosinophilia), bronchopulmonary aspergillosis, polyarteritis nodosa (including Churg-Strauss syndrome), eosinophilic granuloma and eosinophil-related disorders affecting the airways occasioned by drug-reaction.

Compounds of formula I are also useful in the treatment of inflammatory conditions of the skin, for example psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, pemphisus, epidermolysis bullosa acquisita, and other inflammatory conditions of the skin.

Compounds of formula I may also be used for the treatment of other diseases or conditions, in particular diseases or conditions having an inflammatory component, for example, treatment of diseases and conditions of the eye such as conjunctivitis, keratoconjunctivitis sicca, and vernal conjunctivitis, diseases affecting the nose including allergic rhinitis, diseases of the joints such as rheumatoid arthritis and inflammatory bowel disease such as ulcerative colitis and Crohn's disease.

Compounds of formula I are also useful as co-therapeutic agents for use in conjunction with other drug substances for treatment of airways diseases, particularly anti-inflammatory bronchodilatory, antihistamine and anti-tussive drug substances, particularly in the treatment of obstructive or inflammatory airways diseases such as those mentioned hereinbefore, for example as potentiators of therapeutic activity of such drugs or as a means of reducing required dosaging or potential side effects of such drugs. A compound of formula I may be mixed with the other drug in a fixed pharmaceutical composition or it may be administered separately, before, simultaneously with or after the other drug. Accordingly the invention includes a combination of an agent of the invention as hereinbefore described with an anti-inflammatory, bronchodilatory, antihistamine or anti-tussive drug substance, said agent of the invention and said drug substance being in the same or different pharmaceutical composition.

Such anti-inflammatory drugs include LTB4 antagonists, such as BIIL 284, CP-195543, DPC11870, LTB4 ethanolamide, LY 293111, LY 255283, CGS025019C, CP-195543, ONO-4057, SB 209247, SC-53228 and those described in U.S. Pat. No. 5,451,700; LTD4 antagonists, such as montelukast, pranlukast, zafirlukast, accolate, SR2640, Wy-48,252, ICI 198615, MK-571, LY-171883, Ro 24-5913 and L-648051; PDE4 inhibitors such cilomilast (Ariflo® GlaxoSmithKline), Roflumilast (Byk Gulden),V-11294A (Napp), BAY19-8004 (Bayer), SCH-351591 (Schering-Plough), Arofylline (Almirall Prodesfarma), PD189659/PD168787 (Parke-Davis), AWD-12-281 (Asta Medica), CDC-801 (Celgene), SeICID(TM) CC-10004 (Celgene), VM554/UM565 (Vernalis), T-440 (Tanabe), KW-4490 (Kyowa Hakko Kogyo), and those disclosed in WO 92/19594, WO 93/19749, WO 93/19750, WO 93/19751, WO 98/18796, WO 99/16766, WO 01/13953, WO 03/104204, WO 03/104205, WO 03/39544, WO 04/000814, WO 04/000839, WO 04/005258, WO 04/018450, WO 04/018451, WO 04/018457, WO 04/018465, WO 04/018431, WO 04/018449, WO 04/018450, WO 04/018451, WO 04/018457, WO 04/018465, WO 04/019944, WO 04/019945, WO 04/045607 and WO 04/037805; A_{2A} agonists, such as those described in EP 1052264, EP 1241176, EP 409595A2, WO 94/17090, WO 96/02543, WO 96/02553, WO 98/28319, WO 99/24449, WO 99/24450, WO 99/24451, WO 99/38877, WO 99/41267, WO 99/67263, WO 99/67264, WO 99/67265, WO 99/67266, WO 00/23457, WO 00/77018, WO 00/78774, WO 01/23399, WO 01/27130, WO 01/27131, WO 01/60835, WO 01/94368, WO 02/00676, WO 02/22630, WO 02/96462 and WO 03/086408; A_{2B} antagonists, such as those described in WO 02/42298; and beta (β)-2-adrenoceptor agonists, such as albuterol (salbutamol), metaproterenol, terbutaline, salmeterol fenoterol, procaterol, and especially, formoterol, carmoterol and pharmaceutically acceptable salts thereof, and compounds (in free or salt or solvate form) of formula I of WO 0075114, which document is incorporated herein by reference, preferably compounds of the Examples thereof, especially a compound of formula 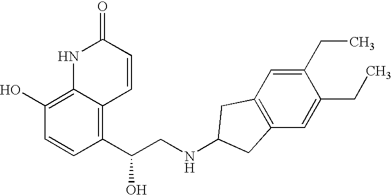
and pharmaceutically acceptable salts thereof, as well as compounds (in free or salt or solvate form) of formula I of WO 04/16601, and also compounds of EP 1440966, JP 05025045, WO 93/18007, WO 99/64035, US 2002/0055651, WO 01/42193, WO 01/83462, WO 02/66422, WO 02/70490, WO 02/76933, WO 03/24439, WO 03/42160, WO 03/42164, WO 03/72539, WO 03/91204, WO 03/99764, WO 04/16578, WO 04/22547, WO 04/32921, WO 04/33412, WO 04/37768, WO 04/37773, WO 04/37807, WO 04/39762, WO 04/39766, WO 04/45618 WO 04/46083 and WO 04/80964.

Such bronchodilatory drugs include anticholinergic or antimuscarinic agents, in particular ipratropium bromide, oxitropium bromide, tiotropium salts and CHF 4226 (Chiesi), and glycopyrrolate, but also those described in EP 424021, U.S. Pat. No. 3,714,357, U.S. Pat. No. 5,171,744, WO 01/04118, WO 02/00652, WO 02/51841, WO 02/53564, WO 03/00840, WO 03/33495, WO 03/53966, WO 03/87094, WO 04/018422 and WO 04/05285.

Such anti-inflammatory and bronchodilatory drugs include dual anti-inflammatory and bronchodilatory drugs especially dual beta-2 adrenoceptor agonist/muscarinic antagonists such as those disclosed in US 2004/0167167, WO 04/74246 and WO 04/74812.

Such co-therapeutic antihistamine drug substances include cetirizine hydrochloride, acetaminophen, clemastine fumarate, promethazine, loratidine, desloratidine, diphenhydramine and fexofenadine hydrochloride, activastine, astemizole, azelastine, ebastine, epinastine, mizolastine and tefenadine as well as those disclosed in JP 2004107299, WO 03/099807 and WO 04/026841.

Combinations of agents of the invention and one or more PDE4 inhibitors, A_{2A} agonists, A_{2B} antagonists, β-2-adrenoreceptor agonists and/or LTD4 antagonists may be used, for example, in the treatment of COPD or, particularly, asthma. Combinations of agents of the invention and one or more anticholinergic or antimuscarinic agents, PDE4 inhibitors, A_{2A} agonists, A_{2B} antagonists, β-2-adrenoreceptor agonists and/or LTB4 antagonists may be used, for example, in the treatment of asthma or, particularly, COPD.

In accordance with the foregoing, the invention also provides a method for the treatment of an inflammatory condition, particularly an inflammatory or obstructive airways disease, which comprises administering to a subject, particularly a human subject, in need thereof an effective amount of a compound of formula I as hereinbefore described. In another aspect the invention provides the use of a compound of formula I as hereinbefore described for the manufacture of a medicament for the treatment of an inflammatory condition, particularly an inflammatory or obstructive airways disease.

The compounds of formula I may be administered by any appropriate route, e.g. orally, for example in the form of a tablet or capsule; parenterally, for example intravenously; by inhalation, for example in the treatment of inflammatory or obstructive airways disease; intranasally, for example in the treatment of allergic rhinitis; topically to the skin, for example in the treatment of atopic dermatitis; or rectally, for example in the treatment of inflammatory bowel disease.

In a further aspect, the invention also provides a pharmaceutical composition comprising as active ingredient a compound of formula I, optionally together with a pharmaceutically acceptable diluent or carrier therefor. The composition may contain a co-therapeutic agent such as a bronchodilatory or anti-inflammatory drug as hereinbefore described. Such compositions may be prepared using conventional diluents or excipients and techniques known in the galenic art. Thus oral dosage forms may include tablets and capsules. Formulations for topical administration may take the form of creams, ointments, gels or transdermal delivery systems, e.g. patches. Compositions for inhalation may comprise aerosol or other atomizable formulations or dry powder formulations.

When the composition comprises an aerosol formulation, it preferably contains, for example, a hydro-fluoro-alkane (HFA) propellant such as HFA134a or HFA227 or a mixture of these, and may contain one or more co-solvents known in the art such as ethanol (up to 20% by weight), and/or one or more surfactants such as oleic acid or sorbitan trioleate, and/or one or more bulking agents such as lactose. When the composition comprises a dry powder formulation, it preferably contains, for example, the compound of formula I having a particle diameter up to 10 microns, optionally together with a diluent or carrier, such as lactose, of the desired particle size distribution and a compound that helps to protect against product performance deterioration due to moisture e.g. magnesium stearate, typically 0.05-1.5%. When the composition comprises a nebulised formulation, it preferably contains, for example, the compound of formula I either dissolved, or suspended, in a vehicle containing water, a co-solvent such as ethanol or propylene glycol and a stabiliser, which may be a surfactant.

The invention includes (A) a compound of formula I in inhalable form, e.g. in an aerosol or other atomisable composition or in inhalable particulate, e.g. micronised, form, (B) an inhalable medicament comprising a compound of formula I in inhalable form; (C) a pharmaceutical product comprising a compound of formula I in inhalable form in association with an inhalation device; and (D) an inhalation device containing a compound of formula I in inhalable form.

Dosages of compounds of formula I employed in practising the present invention will of course vary depending, for example, on the particular condition to be treated, the effect desired and the mode of administration. In general, suitable daily dosages for administration by inhalation are of the order of 0.005 to 10 mg, while for oral administration suitable daily doses are of the order of 0.05 to 100 mg.

The invention is illustrated by the following Examples.

### EXAMPLES 1-5

Compounds of formula I are shown in the following table. Methods for their preparation are described hereinafter. The table also shows mass spectrometry (MH+) data. The examples are in free form.

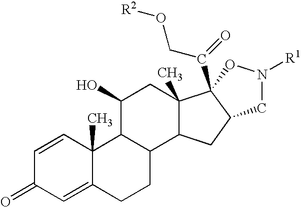

| Ex. | | | |
|---|---|---|---|
| No. | R¹ | R² | MH+ |
| 1 | —CH₂—CH₂—CH₃ | H | 430.3 |
| 2 | —CH₃ | H | 402.1 |
| 3 | —CH₂—CH₂—CH₂—CH₃ | H | 444.2 |
| 4 | 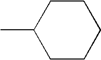 | H | 470.3 |
| 5 | 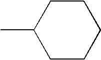 | —CO—CH(CH₃)₂ | 550.1 |

### EXAMPLE 1

To a mixture of acetic acid 2-((4aR,5S,6aS,6bR,9aS)-5-hydroxy-4a,6a-dimethyl-2-oxo-8-propyl-2,4a,4b,5,6,6a,8,9,9a,10,10a,10b,11,12-tetradecahydro-7-oxa-8-aza-pentaleno[2,1-a]phenanthren-6b-yl)-2-oxo-ethyl ester (Example 6) (0.082 g, 0.173 mmol) in methanol (2 ml) is added an aqueous solution of potassium carbonate (0.563 g, 4.08 mmol in 2 ml H₂O). The reaction is stirred for 2 hours, then diluted with water and acidified. The product is extracted into dichloromethane, dried over sodium sulfate and concentrated using a rotary evaporator to yield the desired product, (4aR,5S,6aS,6bR,9aS)-5-Hydroxy-6b-(2-hydroxy -acetyl)-4a,6a-dimethyl-8-propyl-4a,4b,5,6,6a,6b,8,9,9a,10,10a,10b,11,12-tetradecahydro-7-oxa-8-pentaleno[2,1-a]phenanthren-2-one.

### EXAMPLE 2

(4aR,5S,6aS,6bR,9aS)-5-Hydroxy-6b-(2-hydroxy-acetyl)-4a,6a,8-trimethyl-4a,4b,5,6,6a,6b, 8,9,9a,10a,10b,11,12-tetradecahydro-7-oxa-8-aza-pentaleno[2,1-a]phenanthren-2-one is prepared to the compound below using a procedure analogous to that used in Ex. 1.

Acetic acid 2-((4aR,5S,6aS,6bR,9aS)-5-hydroxy-4a,6a,8-trimethyl-2-oxo-2,4a,4b,5,6,6a,8,9, 9a,10,10a,11,12-tetradecahydro-7-oxa-8-aza-pentaleno[2,1-a]phenanthren-6b-yl)-2-oxo-ethyl ester is known from Green et al, J. Med. Chem. (1982) 25: 1492-1495.

### EXAMPLE 3

(4aR,5S,6aS,6bR,9aS)-8-Butyl-5-hydroxy-6b-(2-hydroxy-acetyl)-4a,6a-dimethyl-4a,4b,5,6, 6a,6b,8,9,9a,10,10a,10b,11,12-tetradecahydro-7-oxa-8-aza-pentaleno[2,1-a]phenanthren-2-one is prepared from the compound of Example 7 using a procedure analogous to that used in Example 1.

### EXAMPLE 4

(4aR,5S,6aS,6bR,9aS)-8-Cyclohexyl-5-hydroxy-6b-(2-hydroxy-acetyl)-4a,6a-dimethyl-4a,4b, 5,6,6a,6b,8,9,9a,10,10a,10b,11,12-tetradecahydro-7-oxa-8-aza-pentaleno[2,1-a]-phenan-thren-2-one is prepared from the compound of Example 8 using a procedure analogous to that used in Example 1.

### EXAMPLE 5

A mixture of (4aR,5S,6aS,6bR,9aS)-8-cyclohexyl-5-hydroxy-6b-(2-hydroxy-acetyl)-4a,6a, dimethyl-4a,4b,5,6,6a,6b,8,9,9a,10,10a,10b,11,12-tetradecahydro-7-oxa-8-aza-pentaleno-[2,1-a]phenanthren-2-one (0.054 g, 0.115 mmol) and isobutyric anhydride (0.019 g; 0.121 mmol) in pyridine (0.5 ml) is stirred overnight. The reaction is heated at 35° C. for 2 hours, then a further 0.01 g isobutyric anhydride is added, and the temperature is reduced to room temperature. After 1 hour the reaction is diluted with aqueous HCl and the product is extracted into CH₂Cl₂. The organic layer is dried, and concentrated using a rotary evaporator. The crude product is purified by chromatography, to yield the desired product, isobutyric acid 2-((4aR,5S,6aS,6bR,9aS)-8-cyclohexyl-5-hydroxy-4a,6a-dimethyl-2-oxo-2,4a,4b,5,6,6a,8,9,9a,10,10a,10b,11,12-tetradecahydro-7-oxa-8-aza-pentaleno[2,1-a]-phenanthren-6b-yl)-2-oxo-ethyl ester.

### EXAMPLES 6-8

Compounds of formula II are shown in the following table. Methods for their preparation are described hereinafter. The table includes mass spectrometry (MH+) data. The examples are in free form.

| | |
|---|---|
| | II |
| 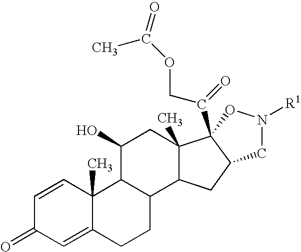 | |

| Ex. | | |
|---|---|---|
| No. | R¹ | MH+ |
| 6 | —CH₂—CH₂—CH₃ | 472.2 |
| 7 | —CH₂—CH₂—CH₂—CH₃ | 486.2 |
| 8 | 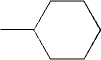 | 512.3 |

### EXAMPLE 6

A mixture of acetic acid 2-((10R,11S,13S)-11-hydroxy-10,13-dimethyl-3-oxo-6,7,8,9,10,11, 12,13,14,15-decahydro-3H-cyclopenta[a]phenanthren-17-yl)-2-oxo-ethyl ester (0.576 g, 1.5 mmol), n-propylhydroxylamine (0.166 g, 1.5 mmol), di-isopropylamine (0.166 g, 1.65 mmol) and paraformaldehyde (0.029 g, 0.97 mmol) in ethanol (25 ml) is heated at 85° C. for 20 hours. The reaction mixture is added to water and the product extracted into dichloromethane. The dichloromethane is dried over sodium sulfate and concentrated using a rotary evaporator to yield the desired product, acetic acid 2-((4aR,5S,6aS,6bR,9aS)-5-hydroxy-4a,6a-dimethyl-2-oxo-8-propyl-2,4a,4b,5,6,6a,8,9,9a,10,10a,1 Ob,11,12-tetrade oxa-8-aza-pentaleno[2,1-a]phenanthren-6b-yl)-2-oxo-ethyl ester.

### EXAMPLE 7

Acetic acid 2-((4aR,5S,6aS,6bR,9aS)-8-butyl-5-hydroxy-4a,6a-dimethyl-2-oxo-2,4a,4b,5,6, 6a,8,9,9a,10,10a,10b,11,12-tetradecahydro-7-oxa-8-aza-pentaleno[2,1-a]phenanthren-6b-yl)-2-oxo-ethyl ester is prepared using a procedure analogous to that used in Example 6.

### EXAMPLE 8

Acetic acid 2-((4aR,5S,6aS,6bR,9aS)-8-cyclohexyl-5-hydroxy-4a,6a-dimethyl-2-oxo-2,4a,4b, 5,6,6a,8,9,9a,10,10a,10b,11,12-tetradecahydro-7-oxa-8-aza-pentaleno[2,1-a]phenanthren-6b-yl)-2-oxo-ethyl ester is prepared using a procedure analogous to that used in Example 6.

1. A compound of formula I 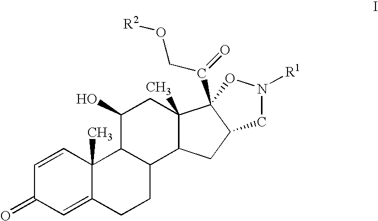
in free or salt form, wherein
R¹ is selected from the group consisting of C₁-C₈-alkyl and C₃-C₈-cycloalkyl; and
R² is selected from the group consisting of hydrogen, C2-C₈-alkylcarbonyl and C₃-C₈-cycloalkylcarbonyl.

2. A compound according to claim 1, wherein
R¹ is selected from the group consisting of C₁-C₈-alkyl and C₃-C₈-cycloalkyl; and
R² is selected from the group consisting of hydrogen and C₂-C₈-alkylcarbonyl.

3. A compound according to claim 2, wherein
R¹ is selected from the group consisting of C₁-C₄-alkyl and C₃-C₆-cycloalkyl, and
R² is selected from the group consisting of hydrogen and C₂-C₄-alkylcarbonyl.

4. A compound according to claim 1, that is
(4aR,5S,6aS,6bR,9aS)-5-Hydroxy-6b-(2-hydroxy-acetyl)-4a,6a-dimethyl-8-propyl-4a,4b,5,6,6a,6b,8,9,9a,10,10a,10b,11,12-tetradecahydro-7-oxa-8-aza-pentaleno[2,1-a]-phenanthren-2-one;
(4aR,5S,6aS,6bR,9aS)-5-Hydroxy-6b-(2-hydroxy-acetyl)-4a,6a,8-trimethyl-4a,4b,5,6,6a,6b, 8,9,9a,10,10a,10b,11,12-tetradecahydro-7-oxa-8-aza-pentaleno [2,1-a]phenanthren-2-one;
(4aR,5S,6aS,6bR,9aS)-8-Butyl-5-hydroxy-6b-(2-hydroxy-acetyl)-4a,6a-dimethyl-4a,4b,5,6, 6a,6b,8,9,9a,10,10a,10b,11,12-tetradecahydro-7-oxa-8-aza-pentaleno [2,1-a]phenanthren-2-one;
(4aR,5S,6aS,6bR,9aS)-8-Cyclohexyl-5-hydroxy-6b-(2-hydroxy-acetyl)-4a,6a-dimethyl-4a,4b, 5,6,6a,6b,8,9,9a,10,10a,10b,11,12-tetradecahydro-7-oxa-8-aza-pentaleno[2,1-a]-phenan-thren-2-one;
Isobutyric acid 2-((4aR,5S,6aS,6bR,9aS)-8-cyclohexyl-5-hydroxy-4a,6a-dimethyl-2-oxo-2,4a,4b,5,6,6a,8,9,9a,10,10a,10b,11,12-tetradecahydro-7-oxa-8-aza-pentaleno[2,1-a]-phenanthren-6b-yl)-2-oxo-ethyl ester;
Acetic acid 2-((4aR,5S,6aS,6bR,9aS)-5-hydroxy-4a,6a-dimethyl-2-oxo-8-propyl-2,4a,4b,5,6,6a,8,9,9a,10,10a,10b,11,12-tetradecahydro-7-oxa-8-aza-pentaleno[2,1-a]phenanthren-6b-yl)-2-oxo-ethyl ester;
Acetic acid 2-((4aR,5S,6aS,6bR,9aS)-8-butyl-5-hydroxy-4a,6a-dimethyl-2-oxo-2,4a,4b,5,6 6a,8,9,9a,10,10a,10b,11,12-tetradecahydro-7-oxa-8-aza-pentaleno[2,1-a]phenanthren-6b-yl)-2-oxo-ethyl ester; or
Acetic acid 2-((4aR,5S,6aS,6bR,9aS)-8-cyclohexyl-5-hydroxy-4a,6a-dimethyl-2-oxo-2,4a,4b, 5,6,6a,8,9,9a,10,10a,10b,11,12-tetradecahydro-7-oxa-8-aza-pentaleno[2,1-a]phenanthren-6b-yl)-2-oxo-ethyl ester.

5. A compound according to claim 1 in combination with another drug substance that is an anti-inflammatory, a bronchodilator, an antihistamine or an anti-tussive drug substance.

6. A compound according to claim 5 wherein said another drug substance is a beta-2 adrenergic receptor agonist.

7. A compound according to claim 1 for use as a pharmaceutical.

8. The use of a compound according to claim 1 for the manufacture of a medicament for the treatment of an inflammatory condition.

9. A pharmaceutical composition comprising as active ingredient a compound according to claim 1, optionally together with a pharmaceutically acceptable diluent or carrier therefor.

10. A process for the preparation of compounds of formula I as defined in claim 1, which comprises:
(i) (A) for the preparation of compounds of formula I wherein R² is hydrogen, hydrolysing a corresponding eser; or
(B) for the preparation of compounds of formula I wherein R² is selected from the group consiting of C₂-C₈-alkylcarbonyl and C₃-C₈-cycloalkylcarbonyl, apprpriately acylating a corrsponding compound of formula I wherein R² is hydrogen; and
(ii) recovering the resultnt compounds of formula I in free or salt form.

11. A compound of formula II 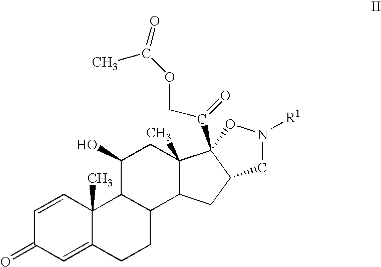
in free or salt form, wherein
R¹ is selected from the group consisting of C₂-C₈-alkyl and C₃-C₈-cycloalkyl.
